(19) United States
(12) Patent Application Publication
(10) Pub. No.: US 2026/0083878 A1
(43) Pub. Date: Mar. 26, 2026

(54) BONE IMPLANT BODY AND METHOD FOR THE PRODUCTION OF SAME

(71) Applicant: Resorba Medical GmbH, Nürnberg (DE)

(72) Inventors: Michael AHLERS, Stein (DE); Gauri TENDULKAR, Nürnberg (DE); Michael GELINSKY, Meißen (DE); Anja LODE, Dresden (DE); Anne BERNHARDT, Dresden (DE); Leonie SCHLICHT, Dresden (DE)

(21) Appl. No.: 19/111,391

(22) PCT Filed: Aug. 19, 2023
(86) PCT No.: PCT/EP2023/073609

§ 371 Date: Mar. 13, 2025

(30) Foreign Application Priority Data
Sep. 14, 2022 (DE) 10 2022 209 599.5

(51) Int. Cl. A61L 27/24; A61L 27/20; A61L 27/54; A61L 27/56

(52) U.S. Cl. CPC A61L 27/24; A61L 27/20; A61L 27/54; A61L 27/56; A61L 2300/414; A61L 2430/02

(57) ABSTRACT

A bone implant body serves to be introduced into a bone defect location to promote bone growth in this bone defect location. It has a main body made of a deformable and porous collagen matrix, which contains at least one main body collagen material of a first proportion in the form of a mineralized collagen and a second proportion in the form of a native collagen, and a depot member, which is completely embedded in the main body such that the depot member is completely surrounded by the collagen matrix of the main body and placed centrally within the main body and contains, as a first constituent, at least one depot material in the form of at least one bioactive substance which promotes bone healing. A transition between the collagen matrix of the main body and the depot member is formed by an interfacial area of the depot member.

### CROSS REFERENCE TO RELATED APPLICATIONS

This application is a United States National Phase Application of International Application PCT/EP2023/073609, filed Aug. 29, 2023, and claims the benefit of priority under 35 U.S.C. § 119 of German Application 10 2022 209 599.5, filed Sep. 14, 2022, the entire contents of which are incorporated herein by reference.

### TECHNICAL FIELD

The invention relates to a bone implant body for being introduced into a bone defect location to promote bone growth in said bone defect location, and a method for the manufacture of such a bone implant body.

### BACKGROUND

Due to illness and/or during medical treatment, bone defects may occur. An example of this is the wound cavity after the clearing away of a bone cyst. The tooth pocket (=alveolus) present after a tooth extraction can also be taken to mean a special case of a bone defect of this type.

If the self-healing capacity of the bone does not lead to healing within an acceptable period of time, the use of replacement materials is necessary. The best results in bone regeneration are achieved with autologous (patient's own) bone tissue. It is the second most commonly transplanted human tissue after blood. In addition to the bone matrix, it also contains stem cells and growth factors. The disadvantages are the low availability as well as the risks and stress for the patient due to the additional surgery required to remove the patient's own bone tissue. Bone tissue from foreign donors (allogeneic) and animals (xenogeneic) is also used as bone replacement material (BRM). Here, cells and bioactive substances are deactivated by processing steps such as decellularisation, deimmunogenisation and/or sterilisation. In addition, biodegradable, artificial bone replacement materials are increasingly being used.

An artificial bone replacement material of this type should be biocompatible, bioresorbable, sterilisable without loss of function and processable into three-dimensional structures of variable size. Furthermore, it should not release cytotoxic substances, allow adhesion and ingrowth of cells and have sufficient mechanical stability.

Collagen-based bone replacement materials, e.g. also in combination with a calcium phosphate mineral phase, simulate the native bone matrix and are therefore suitable bone replacement materials. There are various strategies for introducing a mineral phase into the collagen structure, ranging from simply mixing in ceramic calcium phosphate granules and powders to simultaneous deposition of nano-crystalline hydroxyapatite during collagen fibril reassembly, as is described, for example, in EP 0 945 146 A2 and EP 0 945 147 A2. However, such mineralized collagen may have a high brittleness and disintegrate under mechanical stress. These mechanical properties make it difficult to introduce into a bone defect location.

DE 10 2005 034 421 A1 describes a bioabsorbable mineralized material for filling bone defects, which has a collagen matrix made up of collagen chains that are assembled together, with only the surface of the collagen chains that are assembled together being mineralized.

DE 199 62 090 A1 describes an internally hollow molded body in the geometry of a bone or a part of a bone that serves as a bone replacement. The shaped body is formed from optionally mineralized collagen in the form of a dense network of collagen fibrils, optionally with an additional matrix of calcium phosphate cement, the collagen fibrils being embedded in the matrix of calcium phosphate cement.

The various features of novelty which characterize the invention are pointed out with particularity in the disclosure. For a better understanding of the invention, its operating advantages and specific objects attained by its uses, reference is made to the accompanying drawings and descriptive matter in which preferred embodiments of the invention are illustrated.

### SUMMARY

It is an object of the invention to provide a bone implant body of the type described at the beginning having improved properties compared to the prior art.

This object is achieved by providing a bone implant body according to the features described. The bone implant body according to the invention has a main body made of a deformable and porous collagen matrix which contains at least one main body collagen material consisting of a first proportion in the form of a mineralized collagen and a second proportion in the form of a native collagen, and a depot member, which is completely embedded in the main body such that it is completely surrounded by the collagen matrix of the main body and placed centrally within the main body and contains, as a first constituent, at least one depot material in the form of at least one bioactive substance which promotes bone healing, wherein a transition between the collagen matrix of the main body and the depot member is formed by an interfacial area of the depot member.

Mineralized collagen is understood here to mean collagen that has undergone a further processing step in addition to the actual collagen preparation process, in particular in order to endow it with a mineral phase that promotes bone formation in the bone defect location. A mineralized collagen of this type and a method for the production thereof are known, for example, from EP 0 945 146 A2.

Native collagen is understood here to mean collagen that, in contrast to mineralized collagen, contains essentially no substances foreign to collagen. In this respect, it can also be referred to as mineral-free. It may only contain a small amount of foreign substances originating from the collagen preparation process, but these are not to be classified as significant here. In this respect, the native collagen can in particular also be referred to as pure or as untreated or as original, since it has not undergone any further treatment apart from the actual collagen preparation process.

The collagen matrix and thus also the bone implant body as a whole are deformable, in particular plastically or irreversibly deformable, and preferably also compressible. This allows that only one size of the bone implant body needs to be manufactured and kept in stock, which is very favorable from an economic point of view. The bone implant body, which is only available in one size, can nevertheless be inserted into bone defect locations of very different dimensions due to its advantageous mechanical properties. It can advantageously be adapted to the respective dimensions of the bone defect location concerned due to its plastic deformability and, if necessary, also due to its compressibility. This adaptation is carried out by the surgeon or the attending physician. Plastic deformability is understood to mean in particular the ability of the collagen matrix or also of the bone implant body as a whole to deform or reshape irreversibly under the effect of force after an elasticity limit has been exceeded and to retain this shape after the effect of force has ceased. Such permanent deformation of the bone implant body is favorable because a bone implant body deformed by the surgeon according to the shape and size of the bone defect location and then inserted into the bone defect location should, as far as possible, not exert any restoring forces on the tissue surrounding the bone defect location in order not to impair the healing process. However, such undesirable restoring forces might occur if the deformation is not permanently plastic but only elastic. In addition, the bone implant body has good stability against mechanical stresses.

The bone implant body and also the depot member can in particular have a cylindrical shape. However, other geometries, such as a truncated cone or cuboid shape, are also possible. In the cylindrical embodiment, the bone implant body has a diameter in particular in the range between 0.8 cm and 2.0 cm, preferably in the range between 1.2 cm and 1.6 cm and a length or height in particular in the range between 1.2 cm and 2.0 cm, preferably in the range between 1.4 cm and 1.8 cm and preferably of 1.6 cm. The volume of the depot member is in particular between 10% and 40%, preferably between 15% and 30% of the total volume of the bone implant body.

Mineralized collagen does not, or at least not readily, have these desired favorable mechanical properties such as deformability and/or compressibility. Mineralized collagen even shows a rather opposite behavior. It can be firm, stiff and brittle. On the other hand, it favors bone growth due to the embedded mineral phase. In order for the main body and also the bone implant body as a whole to have the desired mechanical properties, such as deformability, compressibility and the like, the main body collagen material also contains native collagen in addition to the mineralized collagen. The latter provides these desired mechanical properties. In addition, native and mineralized collagen, like all collagen, have a good haemostyptic (=blood-stopping) effect, which promotes wound healing.

The additional porosity of the collagen matrix allows new (bone) cells to grow into the pores of the collagen matrix, thus favoring the bone healing process.

The bioactive substance of the depot material serves to stimulate the formation of new bone. In particular, it is a growth factor that promotes bone growth. Like the mineralized collagen, it supports bone formation in the bone defect location. In particular, the depot member may also contain a combination of at least two bioactive substances that promote bone healing as depot material.

It has been recognized that after introducing collagen material loaded with a bioactive substance into a bone defect location, there is often a very rapid release of the bioactive substance. Instead, to allow a longer lasting, controlled release of the active component, the depot member with the bioactive substance of the depot material is fully embedded in the main body. The bone implant body according to the invention therefore in particular does not have a layered structure. Rather, the depot member is completely surrounded by the collagen matrix of the main body. It is placed centrally within the main body. In this way, the depot material is also delivered as uniformly as possible in all directions outwards to the bone defect location.

The transition formed by the interfacial area of the depot member between the collagen matrix of the main body and the depot member is in particular not smooth, but instead preferably sharp or abrupt. The interfacial area is in particular the outer boundary wall of the depot member. This abrupt transition results in particular from the advantageously separate production of the depot member, which is independent of the production of the collagen matrix.

Advantageous embodiments of the bone implant body according to the invention result from the features described.

A favorable embodiment is one in which the collagen matrix of the main body consists exclusively of the main body collagen material. Its structure is then particularly simple. It can be produced with relatively little effort and, in particular, on an industrial scale.

According to a further favorable embodiment, the main body collagen material is a mixture, in particular a homogeneous mixture, of mineralized collagen and native collagen. Another combination of the mineralized collagen and the native collagen is also possible, in particular. However, it is advantageous with regard to the best possible and uniform deformability and compressibility of the main body and also of the bone implant body as a whole if the mineralized collagen and the native collagen are mixed as well as possible.

According to a further favorable embodiment, in the main body collagen material, a volume ratio between the mineralized collagen and the native collagen is in the range between 20 to 80 and 80 to 20, preferably between 25 to 75 and 75 to 25 and preferably between 30 to 70 and 70 to 30 and more preferably 60 to 40.

According to a further favorable embodiment, the depot member is a separately storable component. In particular, the depot member has a moisture content of at most 20%.

According to a further favorable embodiment, the depot member contains a depot collagen material as a further (in particular second) component. The depot collagen material consists in particular only of mineralized collagen or of both mineralized collagen and native collagen. In addition to the depot material as the first component, the depot member contains the depot collagen material as a further or second component. It can thus be embedded very well in the collagen matrix of the main body and also provides a contribution of mineral phase for bone growth.

According to another favorable embodiment, the bone implant body contains a retarding agent which delays the release of the depot material. The retarding agent is in particular heparin. The retarding agent is preferably present in the depot member, in particular as a third component. However, it can also be present in the main body, in particular additionally or alternatively. The retarding agent strengthens the binding of the at least one bioactive substance of the depot material within the bone implant body and thus slows down the release of the at least one bioactive substance. By means of the retarding agent, the release of the depot material can be adjusted in a targeted manner, in particular slowed down, in order to thereby maintain the effect of the depot material promoting bone healing over a longer period of time.

According to a further favorable embodiment, the mineralized collagen contains a mineral phase in the form of mineral crystallites, wherein the mineral crystallites have a maximum extension of in particular at most 1 μm (micrometer), and preferably at most 100 nm (nanometers), and the mineral crystallites are preferably calcium phosphate crystallites, most preferably hydroxyapatite crystallites. The mineral crystallites are in particular arranged inside collagen fibrils. However, they can also surround the collagen fibrils, in particular on the outside. Preferably, the mineral crystallites are aligned along the orientation of the collagen fibrils. The mineral crystallites have a minimum extension of in particular at least 0.5 nm.

According to another favorable embodiment, the mineralized collagen is crosslink-free. By “crosslink-free” is to be understood here that a small amount of natural crosslinking (or basic crosslinking) may be present, but no further externally provided crosslinking, such as chemical or enzymatic crosslinking. Such further crosslinking, which can also be referred to as synthetic, is formed in particular by using an artificial crosslinker. This can be done, for example, by means of a subsequent chemical (transverse) crosslinking process. The crosslink-free mineralized collagen relevant here has been produced without the use of such an artificial crosslinker. It is firmer, more brittle and stiffer than mineralized collagen that has been specifically crosslinked by means of an artificial crosslinker. In addition, the crosslink-free mineralized collagen relevant here may be unstable to mechanical deformation. In this respect, it has less favorable mechanical properties than synthetically crosslinked mineralized collagen, but this is deliberately accepted because an artificial crosslinker might have an undesirable effect on the depot material, so that the effect of the latter of promoting bone healing is impaired and, in the worst case, even completely lost. In order to prevent this, the use of an artificial crosslinker is dispensed with here. To obtain the desired mechanical properties, at least the main body collagen material also contains native collagen in addition to the advantageously crosslink-free mineralized collagen.

According to yet another favorable embodiment, at least one collagen selected from the group consisting of mineralized collagen and native collagen is an atelocollagen, in particular an atelocollagen of equine origin, and preferably a type 1 atelocollagen of equine origin. It is thus possible that the mineralized collagen or the native collagen is such an atelocollagen. It is also possible for both to be such. An atelocollagen of this type has a particularly high biocompatibility. The human body can degrade or resorb it very well. Collagen of equine origin is obtained in particular from horse tendons.

According to a further favorable embodiment, the bioactive substance promoting bone healing is at least one active component from the group of VEGF, TGF-β, BMP-2, BMP-4, BMP-7, another member of the BMP family, PDGF-BB, another member of the PDGF family, SDF-1α, EGF, bFGF1, bFGF2, dexamethasone, hydrocortisone and cholecalciferol or contains such an active component. Likewise, the bioactive substance promoting bone healing may be at least one active component mixture from the group of human blood plasma (e.g. FFP), human serum, human platelet concentrate (e.g. PL, PRGF, PRP) and hypoxia-conditioned medium concentrate or contain such an active component mixture. VEGF, TGF-β, BMP-2, BMP-4, BMP-7, PDGF-BB, SDF-1α, EGF, bFGF1 and bFGF2 are proteins that can be produced in particular biotechnologically with the aid of genetically modified organisms. The blood plasma, serum and platelet concentrate also mentioned can be obtained in particular from human blood. The hypoxia-conditioned medium concentrate is to be produced in particular from cell cultures. In principle, other active components and/or active component mixtures are also possible. For example, the bioactive substance promoting bone healing may be an active component from the group consisting of members of the TGF-β superfamily, members of the BMP family, the growth differentiation factors (GDFs), ADMP-1, members of the fibroblast growth factor family, members of the hedgehog protein family, members of the insulin-like growth factor (IGF) family, members of the platelet-derived growth factor (PDGF) family, members of the interleukin (IL) family and members of the colony stimulating factor (CSF) family. The bioactive substance promoting bone healing is in particular a growth factor.

According to another favorable embodiment, at least one component from the group consisting of the main body and the depot member contains an antimicrobial, antibiotic, wound-healing or anti-inflammatory substance. It is therefore possible that the main body and/or the depot member contain/contains at least one of these active components. This antimicrobial substance helps to prevent and/or fight infections. It is preferably a locally tolerated antiseptic, such as polihexanide, octenidine, silver (in particular silver compounds or silver particles), iodine derivatives, chlorhexidine, triclosan or the like. Likewise, an antibiotically active substance suitable for local application, such as gentamicin, metronidazole, vancomycin, clindamycin or the like, may be used.

It is another object of the invention to provide a method for the manufacture of a bone implant body of the type referred to at the beginning and having improved properties compared to the prior art.

In order to achieve the object relating to the method, a method according to the features provided. In the method according to the invention, the depot member is first produced separately. Then, during the production of the collagen matrix of the main body, the depot member, which is in particular capable of being stored, is introduced as a finished partial component into a collagen-containing matrix basic substance.

The collagen-containing matrix basic substance is intended in particular to form the collagen matrix of the main body.

The successive and essentially mutually independent production of the depot member and the collagen matrix of the main body simplifies the manufacture. In addition, it is possible to produce the depot member in advance and store it until it is needed to manufacture the complete bone implant body. This also simplifies the manufacture and in particular the logistics to be provided therefor.

The method according to the invention and its embodiments present substantially the same advantages that have already been described in connection with the bone implant body according to the invention and the embodiments thereof.

A number of advantageous embodiments of the method according to the invention follow from the features provided.

A favorable embodiment is one in which the depot member is produced by freeze-drying a collagen-containing depot basic substance to which the at least one depot material has been added.

According to a further favorable embodiment, the collagen matrix of the main body is produced after the depot member has been introduced into the collagen-containing matrix basic substance by freeze-drying the collagen-containing matrix basic substance. Both native collagen and mineralized collagen can advantageously be processed into porous structures by freeze-drying.

### BRIEF DESCRIPTION OF THE DRAWINGS

### In the Drawings:

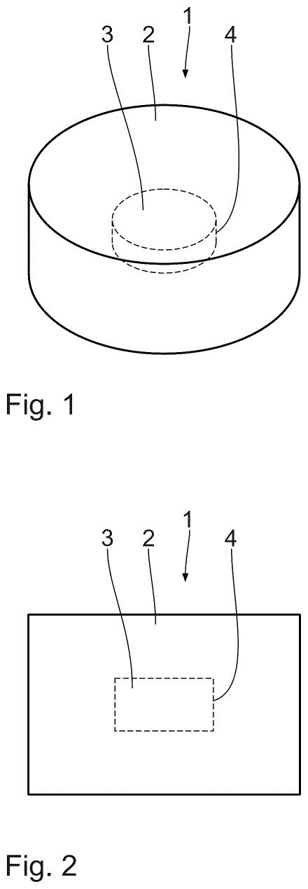

FIG. 1 is a perspective schematic view showing an embodiment example of a bone implant body intended for being introduced into a bone defect location with a depot member embedded in a collagen matrix;

FIG. 2 is a schematic cross-sectional view showing the bone implant body according to FIG. 1;

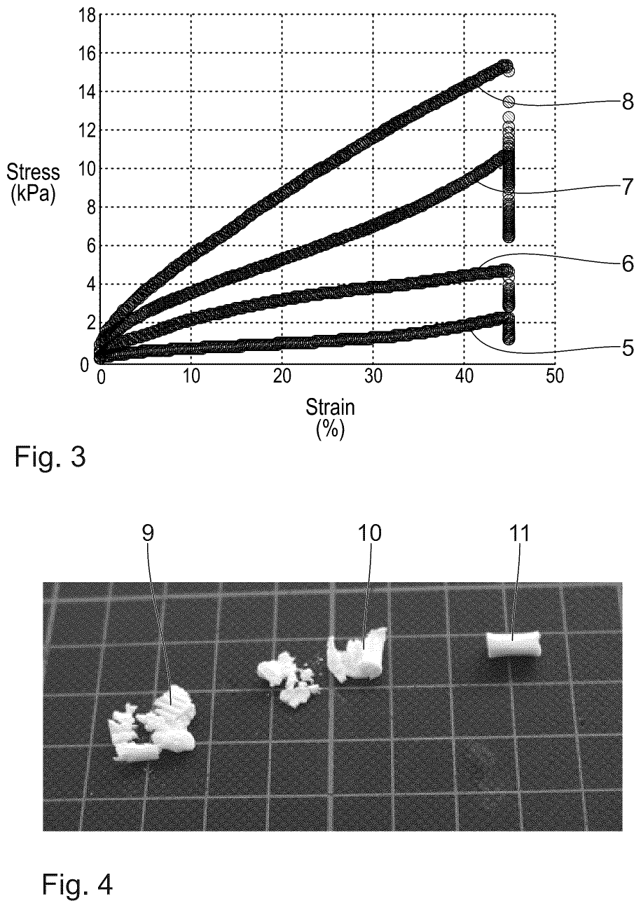

FIG. 3 is a diagram with pressure-compression sequences of different collagen-containing molded bodies;

FIG. 4 is a view showing embodiment examples of different collagen-containing molded bodies after having carried out a brittleness test;

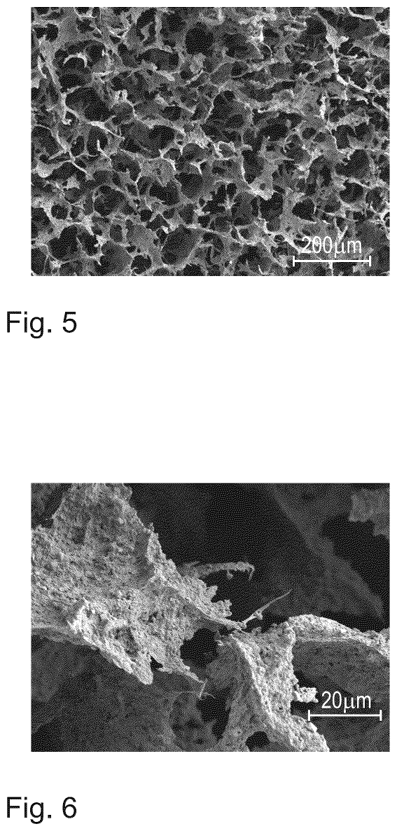

FIG. 5 is a SEM image of the pore structure of a molded body of mineralized collagen;

FIG. 6 is another SEM image of the pore structure of a molded body of mineralized collagen;

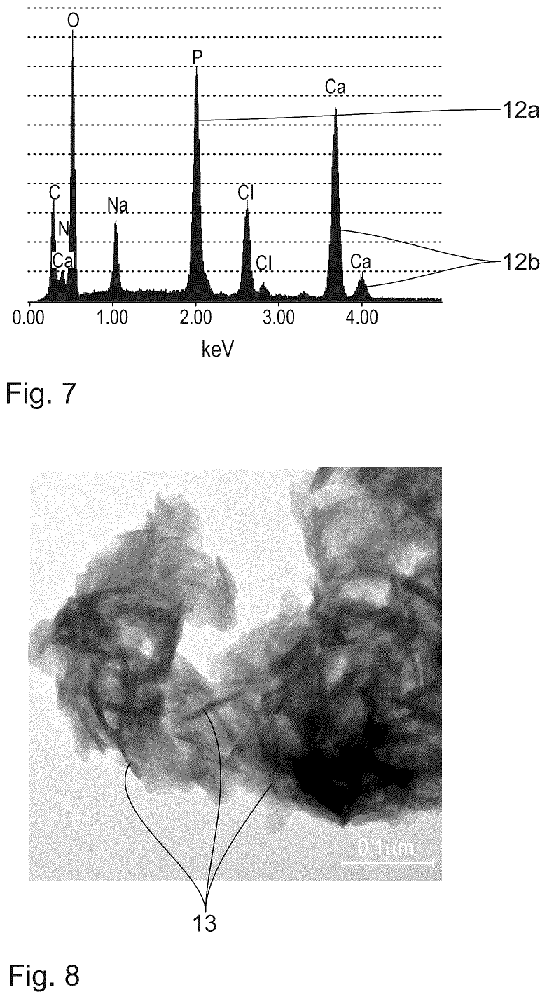

FIG. 7 is an EDX spectrum of mineralized collagen;

FIG. 8 is a TEM image of mineralized collagen;

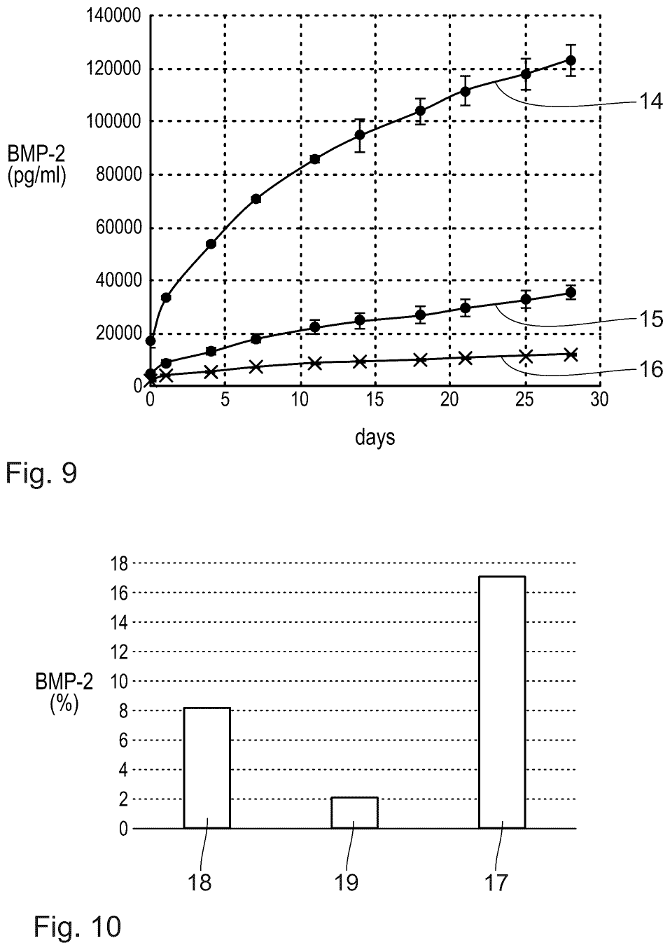

FIG. 9 is a diagram showing the time courses of the release of the bioactive substance BMP-2 from depot members containing different mineralized collagen materials;

FIG. 10 is a diagram showing percentage releases of the bioactive substance BMP-2 from depot members containing different mineralized collagen materials over a period of 14 days; and

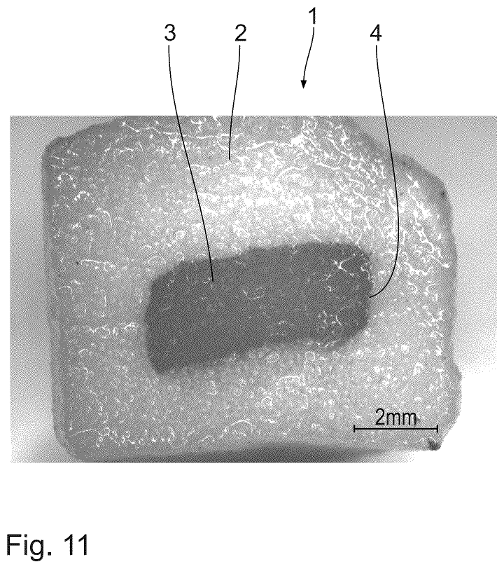

FIG. 11 is a sample of the bone implant body according to FIG. 1 in a photographic cross-sectional illustration comparable to the schematic cross-sectional illustration of FIG. 2.

### DESCRIPTION OF PREFERRED EMBODIMENTS

Referring to the drawings, mutually corresponding parts are marked with the same reference numerals in FIGS. 1 to 11. Details of the embodiment examples explained in more detail below may also constitute an invention in their own right or form part of an object of an invention.

FIGS. 1 and 2 each show a schematic line drawing of an embodiment example of a bone implant body 1 which is intended to be introduced into a bone defect location. In the embodiment example shown, it has a cylindrical shape. Other shapes not shown, such as a truncated cone shape, are also possible. The bone implant body 1 has a main body 2 made of a collagen matrix and a depot member 3 fully embedded in the collagen matrix of the main body 2. The transition between the depot member 3 and the collagen matrix of the main body 2 is not smooth but abrupt. It is formed by an outer interfacial area 4 of the depot member 3. In FIG. 11 a photograph of a realized sample of the bone implant body 1 is shown. The photograph shows approximately the same cross-sectional illustration as the schematic line drawing according to FIG. 2. In the sample shown in FIG. 11, the depot member 3 is colored to make it easier to see.

The main body collagen material of the collagen matrix of the in particular plastically deformable and preferably also compressible main body 2 consists of a homogeneous mixture of mineralized collagen as the first proportion or constituent and of native collagen as the second proportion or constituent. In the embodiment example shown, the volume ratio of mineralized collagen to native collagen is 60 to 40. Other volume ratios not shown, such as 80 to 20, are also possible.

The depot member 3 has a depot collagen material as one constituent, which is composed of 100% mineralized collagen. A further constituent of the depot member 2 is a depot material that promotes bone healing, which in the embodiment shown is the bioactive substance BMP-2, a growth factor. In addition, the depot member 3 contains as a further constituent a retarding agent which delays the release of the growth factor BMP-2 and which, in the embodiment example, is heparin.

Both the main body 2 and the depot member 3 each consist either entirely or at least to a substantial proportion of collagen-containing material. The collagen matrix of the main body 2 consists of a mixture of mineralized collagen and native collagen. The collagen-containing material of the depot member 3, on the other hand, consists only of mineralized collagen, to which, however, the growth factor BMP-2 and the retarding agent heparin have also been added. The production of these different collagen-containing materials is described below.

### Preparation of a Mineral Collagen Suspension

The production and also the use of mineralized collagen is described, for example, in DE 10 2004 044 102 B4 and EP 0 945 146 A2. The method used here is based on these well-known methods.

In a 2 l Erlenmeyer flask, dissolve 1 g acid-soluble collagen (type 1 atelocollagen of equine origin from Resorba Medical GmbH in Nuremberg, Germany) in 1 l 10 mmol/l HCl (prepared from 100 ml 0.1 mol/l HCl and 900 ml deionized water) while stirring vigorously (large stirring fish/magnetic stirrer). Continue stirring vigorously and add in sequence:
1) 180 ml 0.1 mol/1 CaCl2 solution
2) 120 ml 2 mol/l NaCl solution
3) 168 ml 0.5 mol/l TRIS buffer solution (pH=7.5)
4) 500 ml deionized water
5) 22.6 ml phosphate buffer according to Sörensen (0.5 mol/l; pH=7.4)

When adding the phosphate buffer, a milky turbidity of the solution occurs due to precipitating calcium phosphates. Finally, fill up to 2.0 l with deionized water. Then stir vigorously again for about 1 min, close the flask with a lid and place it in a heat bath tempered to 37° C. for 12-24 h.

The mineralized collagen is separated by centrifugation. For this purpose, the gelatinous precipitate is stirred vigorously and then filled into centrifuge tubes (e.g. plastic tubes for 30 ml). Centrifuge for 23 min at 5200 rpm in a refrigerated centrifuge at 4° C. to prevent heating of the product and then pour off the supernatant.

Now the suspension is added and the centrifugation is repeated until the entire preparation has been treated accordingly. After pouring off the supernatant for the last time, the pellets are taken out of the tubes with a spoon spatula and collected in a 125 ml beaker. While stirring with a heavy stirring fish, just enough deionized water is now added drop by drop until a pourable mineral-collagen suspension is obtained.

### Preparation of a Mineral Collagen Suspension With Heparin

The preparation is essentially analogous to the preparation of the mineral collagen suspension already described. The only difference is that after adding the phosphate buffer, a defined amount of heparin (5 mg/g collagen to 150 mg/g collagen layers), pre-dissolved in deionized water, is also added. All other steps remain the same. At the end, a mineral-collagen suspension with heparin is obtained.

### Preparation of a Native Collagen Suspension

Acid soluble collagen (type 1 atelocollagen of equine origin from Resorba Medical GmbH in Nuremberg, Germany) is dissolved in 0.1 M HCl or 6-13 mM acetic acid at a concentration between 1 mg/ml and 35 mg/ml.

The different collagen suspension variants (mineral collagen suspension without and with heparin, native collagen suspension) are filled into blister molds as individual components or as a mixture in different ratios, for example with a volume ratio of mineralized collagen to native collagen of 60 to 40 or of 80 to 20, and freeze-dried.

The bone implant body 1 with the depot member 3 is manufactured in two steps:

First, the depot member 3 is produced from the collagen suspension variants described above, in the embodiment example only from mineral collagen suspension with heparin, and loaded with a depot material in the form of a bioactive substance, in the embodiment example in the form of the growth factor BMP-2. The still liquid collagen-containing depot basic substance formed in this way is filled into a blister mold. The basic depot substance is then freeze-dried, resulting in the depot member 3 as an intermediate product that is in particular storable in this state. The depot member 3 has a (residual) moisture content of only about 10 % to 15 % and can therefore be stored very well.

The collagen-containing matrix basic substance of the collagen matrix of the main body 2 is also produced from the collagen suspension variants described above, in the embodiment example as a mixture of the mineral collagen suspension and the native collagen suspension in the respective desired volume ratio, e.g. of 60 to 40 or of 80 to 20. In another embodiment, however, the matrix basic substance can also be produced only from native collagen suspension. This collagen-containing matrix basic substance is then filled in liquid form into a blister mould which has a larger diameter than the shape of the depot member 3. The previously produced depot member 3 is either pressed into the liquid matrix basic substance, wherein the matrix basic substance flows around the depot member 3, or the depot member 3 is placed on a blister mold half-filled with matrix basic substance, which blister mold is then filled with matrix basic substance. Both variants lead to the complete inclusion of the depot member 3 within the collagen matrix of the main body 2 after the subsequent freeze-drying and the end product is available in the form of the bone implant body 1.

In the following, with reference to FIGS. 3 to 10, properties of this bone implant body 1 or of the collagen-containing materials used for the manufacture thereof are described.

FIG. 3 shows a diagram with the results of compression tests on various truncated cone-shaped molded bodies containing collagen. The tests have been carried out on molded bodies in an essentially dry state with the aid of a Zwick materials testing machine equipped with a 1 kN load cell. All molded bodies were examined at room temperature. They were compressed to up to 40 % of their original length (of e.g. 1.6 cm). In the diagram in FIG. 3, the curves of the applied pressure (“stress”), which corresponds to the compression force, are in each case plotted against the resulting mechanical stretch (“strain”) or compression of the molded bodies. Curve 5 shows the curve for a molded body made of native collagen only, curve 6 shows the curve for a molded body made of a mixture of mineralized collagen and native collagen with a volume ratio of 60:40, curve 7 shows the curve for a molded body made of a mixture of mineralized collagen and native collagen with a volume ratio of 80:20 and curve 8 shows the curve for a molded body made of non-crosslinked mineralized collagen only. Molded bodies made only of native collagen have the highest compressibility (curve 5). However, molded bodies made from mixtures of mineralized and native collagen (curves 6 and 7) also have almost as good compressibility as molded bodies made only from native collagen, and in any case considerably better compressibility than molded bodies made only from non-crosslinked mineralized collagen (curve 8). In this respect, molded bodies made of mixtures of mineralized and native collagen show a lower resistance to compression. A proportion of only 20% of native collagen already significantly reduces the pressure required for 25% compression compared to a molded body consisting only of non-crosslinked mineralized collagen. The addition of native collagen consequently increases the compressibility and deformability of the molded body.

FIG. 4 shows the results of brittleness tests carried out on molded bodies made of different collagen materials. In this test, the cylindrical molded bodies, each having a length of 8 mm and a diameter of 6 mm, were subjected to shear forces. For this purpose, they were rolled back and forth 2 cm each on a flat surface under pressure load. This procedure was repeated three times. A metal plate having a grooved surface (8 grooves per cm, indentation 0.3 mm) was used to exert the shear compression load. The compression load was approx. 1.2 N. The molded body 9 shown in FIG. 4 on the left consisted of non-crosslinked mineralized collagen, the molded body 10 shown in the middle consisted of crosslinked mineralized collagen and the molded body 11 shown on the right consisted of a mixture of mineralized collagen and native collagen having a volume ratio of 60:40. The molded bodies 9 and 10 have disintegrated into individual parts, whereas the molded body 11 has remained intact as a whole. The addition of native collagen consequently reduces the brittleness of the molded body.

Overall, the addition of native collagen therefore has a favorable effect on the mechanical properties of the molded bodies examined and thus also of the bone implant body 1.

FIGS. 5 and 6 show images of the structure of molded bodies of mineralized equine collagen taken by a scanning electron microscope (SEM). These molded bodies have a uniform, interconnecting pore structure having pore diameters of up to 100 μm. This pore structure is not affected by the insertion of the depot member 3 into the collagen matrix of the main body 2. Nothing can be seen that could affect the stability of the molded body. The pores in the collagen material of the bone implant body 1 are favorable as they allow the ingrowth of new bone cells.

FIG. 7 shows a spectrum of an energy dispersive X-ray spectroscopy (EDX) analysis of a region of a sample of mineralized collagen. Peaks 12a and 12b demonstrate that the sample under investigation has a higher content of phosphorus (peak 12a) and calcium (peak 12b) than would be the case for a sample of native collagen. The sample under investigation contains hydroxyapatite.

FIG. 8 shows an image of mineralized collagen taken by transmission electron microscopy (TEM). The morphology of the hydroxyapatite crystallites 13 can be seen. The dimension of the hydroxyapatite crystallites 13 is in the nm range. The longitudinal extension of the hydroxyapatite crystallites 13 is about 100 nm.

The diagrams in FIGS. 9 and 10 relate to the release of the depot material stored in the depot member 3 in the form of the growth factor BMP-2. FIG. 9 shows the time courses of the cumulative amounts of released BMP-2 in depot members 3 with different mineralized collagen materials. The cumulative amounts of BMP-2 released (y-axis) are plotted against time (x-axis) in each case. Curve 14 shows the release curve for a depot member 3 of mineralized collagen of bovine origin, curve 15 the release curve for a depot member 3 of mineralized collagen of equine origin and curve 16 the release curve for a depot member 3 of mineralized collagen of equine origin with heparin as a retarding agent.

FIG. 10 shows the cumulative percentage releases of the growth factor BMP-2 from depot members with different mineralized collagen materials over a period of 14 days. The right bar 17 shows the cumulative percentage release rate for a depot member 3 of mineralized collagen of bovine origin, the left bar 18 the cumulative percentage release rate for a depot member 3 of mineralized collagen of equine origin and the middle bar 19 the cumulative percentage release rate for a depot member 3 of mineralized collagen of equine origin with heparin as a retarding agent.

FIGS. 9 and 10 show that mineralized collagen of equine origin can bind BMP-2 significantly better than native collagen (not shown in FIGS. 9 and 10) and mineralized collagen of bovine origin. The addition of the retarding agent heparin further increases the binding capacity. The higher the binding capacity, the lower the release rate. A bone implant body 1 with the lowest possible BMP-2 release rate is favorable, as then the growth factor BMP-2 can support bone healing over a longer period of time. The release rate can be set to a desired value by the amount of retarding agent added.

While specific embodiments of the invention have been shown and described in detail to illustrate the application of the principles of the invention, it will be understood that the invention may be embodied otherwise without departing from such principles. IN THE CLAIMS:

1. A bone implant body for being introduced into a bone defect location for promoting bone growth in said bone defect location, the bone implant body comprising:
a main body made of a deformable and porous collagen matrix which contains at least one main body collagen material consisting of a first proportion comprising a mineralized collagen and a second proportion comprising a native collagen; and
a depot member which is completely embedded in the main body such that the depot member is completely surrounded by the collagen matrix of the main body and placed centrally within the main body and contains, as a first constituent, at least one depot material comprising at least one bioactive substance which promotes bone healing, wherein a transition between the collagen matrix of the main body and the depot member is formed by an interfacial area of the depot member.

2. A bone implant body according to claim 1, wherein the collagen matrix of the main body consists exclusively of the main body collagen material.

3. A bone implant body according to claim 1, wherein the main body collagen material is a mixture of the mineralised collagen and the native collagen.

4. A bone implant body according to claim 1, wherein the main body collagen material comprises a volume ratio between the mineralized collagen and the native collagen in a range between 20 to 80 and 80 to 20.

5. A bone implant body according to claim 1, wherein the depot member is a separately storable component and has a moisture content of at most 20%.

6. A bone implant body according to claim 1, wherein the depot member contains a depot collagen material as a further constituent, and the depot collagen material consists of mineralized collagen only or of both mineralized collagen and native collagen.

7. A bone implant body according to claim 1, wherein the bone implant body contains a retarding agent which delays the release of the depot material, and the retarding agent is heparin.

8. A bone implant body according to claim 1, wherein the mineralized collagen contains a mineral phase in the form of mineral crystallites, wherein the mineral crystal-lites have a maximum extension of at most 1 μm.

9. A bone implant body according to claim 1, wherein the mineralized collagen is crosslink-produced without the use of an artificial crosslinker and therefore free of synthetic crosslinking.

10. A bone implant body according to claim 1, wherein at least one collagen selected from the group consisting of the mineralized collagen and the native collagen is an atelocollagen.

11. A bone implant body according to claim 1, wherein the bioactive substance promoting bone healing is at least one active component from the group of VEGF, TGF-β, BMP-2, BMP-4, BMP-7, another member of the BMP family, PDGF-BB, another member of the PDGF family, SDF-1α, EGF, bFGF1, bFGF2, dexamethasone, hydrocortisone and cholecalciferol or contains such an active component, or the bone healing-promoting bioactive substance is at least one active component mixture selected from the group consisting of human blood plasma, human serum, human platelet concentrate and hypoxia-conditioned medium concentrate, or contains such an active component mixture.

12. A bone implant body according to claim 1, wherein at least one component from the group consisting of the main body and the depot member contains an antimicrobial, antibiotically active, wound-healing or anti-inflammatory substance.

13. A method for the manufacture of a bone implant body for being introduced into a bone defect location for promoting bone growth in said bone defect location, the bone implant body comprising a main body made of a deformable and porous collagen matrix which contains at least one main body collagen material consisting of a first proportion in the form of a mineralized collagen and a second proportion in the form of a native collagen; and a depot member which is completely embedded in the main body such that the depot member is completely surrounded by the collagen matrix of the main body and placed centrally within the main body and contains, as a first constituent, at least one depot material in the form of at least one bioactive substance which promotes bone healing, wherein a transition between the collagen matrix of the main body and the depot member is formed by an interfacial area of the depot member, wherein
the depot member is first produced separately, and
the depot member is subsequently introduced as a finished partial component into a collagen-containing matrix basic substance during the production of the collagen matrix of the main body.

14. A method according to claim 13, wherein the depot member is produced by freeze-drying a collagen-containing depot basic substance to which the at least one depot material has been added.

15. A method according to claim 13, wherein the collagen matrix of the main body is produced, after the depot member has been introduced into the collagen-containing matrix basic substance, by means of freeze-drying of the collagen-containing matrix basic substance.

16. A bone implant body according to claim 1, wherein the main body collagen material, a volume ratio between the mineralized collagen and the native collagen is in the range between 25 to 75 and 75 to 25.

17. A bone implant body according to claim 1, wherein the main body collagen material comprises a volume ratio between the mineralized collagen and the native collagen in a range between 30 to 70 and 70 to 30.

18. A bone implant body according to claim 1, wherein the main body collagen material comprises a volume ratio between the mineralized collagen and the native collagen of 60 to 40.

19. A bone implant body according to claim 8, wherein the mineral crystallites have a maximum extension of at most 100 nm, and the mineral crystallites comprise calcium phosphate crystallites.

20. A bone implant body according to claim 10, wherein the at least one collagen selected from the group consisting of the mineralized collagen and native collagen comprises an atelocollagen of equine origin.
